(19) United States
(12) Plant Patent Application Publication
(10) Pub. No.: US 2005/0204441 P1
(43) Pub. Date: Sep. 15, 2005

(54) Zantedeschia plant named 'Goldilocks'

(50) ZANTEDESCHIA SP.; GOLDILOCKS

(75) Inventor: Beverley Joe, Auckland (NZ)

Correspondence Address: JONDLE & ASSOCIATES P.C., 858 HAPPY CANYON ROAD SUITE 230, CASTLE ROCK, CO, 80108, US

(73) Assignee: Green Harvest Pacific Holdings LTD

(21) Appl. No.: 10/798,956

(22) Filed: Mar. 11, 2004

(51) Int. Cl. A01H 5/00

(52) U.S. Cl. PLT/263

(57) ABSTRACT

A Zantedeschia plant particularly distinguished by its compact size, yellow spathe and maculated leaves.

### GENUS AND SPECIES

Zantedeschia spp.

### VARIETY DENOMINATION

‘Goldilocks’

### BACKGROUND OF THE NEW PLANT

The present invention comprises a new and distinct cultivar of Calla Lily, botanically known as Zantedeschia spp. and hereinafter referred to by the cultivar name ‘Goldilocks’. The new cultivar is a seedling from a combination of Manukau Horticulture Ltd breeding program and Green Harvest Pacific Holdings breeding program in Auckland, New Zealand. The plants were produced by open pollination, originally from selected “dwarf” lines, then a single selection of unknown parentage. Material was crossed in the 1992/1993 season and the selection was made in 1995. The breeder's reference for the variety was 9422. Stable material was produced in 1997 through in vitro propagation.

‘Goldilocks’ is a dwarf Zantedeschia, characterized by its high number of yellow flowers and the good proportions of the plant and flowers. These features make ‘Goldilocks’ an excellent pot-plant variety.

‘Goldilocks’is similar to ‘Black Magic’ but differs in overall plant size by being smaller and shorter. The plant tends to produce all its flowers at similar stages, rather than staggered like ‘Black Magic’. ‘Goldilocks’ leaves typically exhibit some degree of interveinal yellowing or exaggerated green coloration in vein.

The variety is maintained in breeding as an elite parent and maintained by reproduction through tissue culture. Clones of ‘Goldilocks’ (produced by tissue culture) are identical to the parent plant. No off-types have been observed during trials or production.

The new cultivar was selected because its spathe height is in proportion to leaf length and the average spathe length is 20-30% shorter than existing pot-plant cultivars such as ‘Apricot Glow’ (U.S. Plant Pat. No. 7,653). Later trials showed ‘Goldilocks’ had a high resistance to disease (fungal and bacterial).

### DESCRIPTION OF PHOTOGRAPH

This new Zantedeschia plant is illustrated by the accompanying photographs which show blooms, buds, and foliage of the plant in full color. The colors shown being as true as can be reasonably obtained by conventional photographic procedures.

FIG. 1 shows close-up of plant with ruler (height).

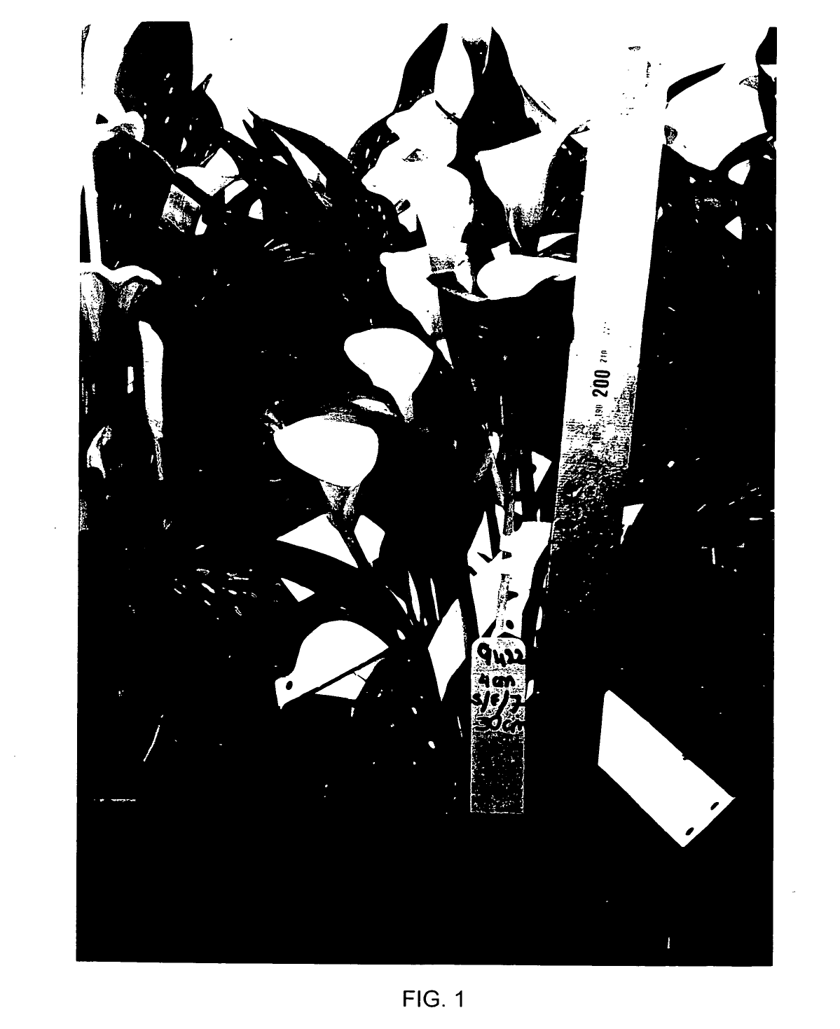

FIG. 2 shows overhead view of habit of plant and leaf shape.

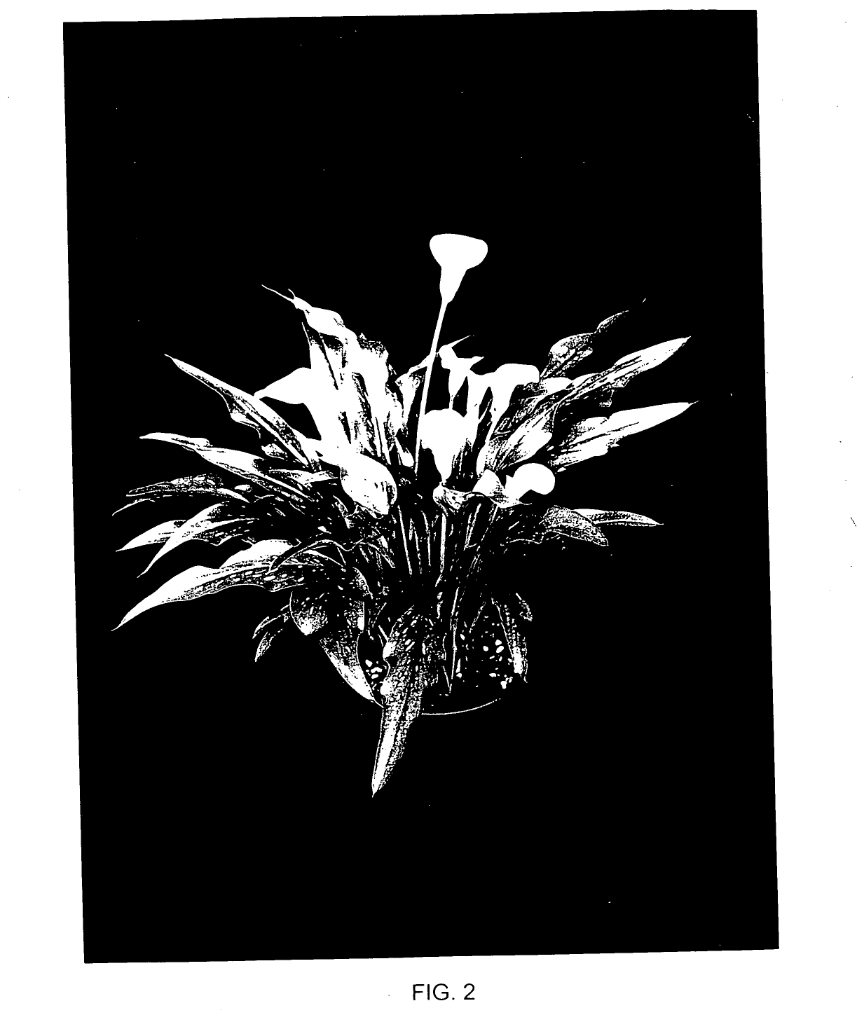

### DESCRIPTION OF THE NEW CULTIVAR

The following detailed descriptions set forth the distinctive characteristics of ‘Goldilocks’. The data which defines these characteristics were collected from asexual reproductions carried out in New Zealand. The plant history was taken on 2 year old plants based on a 4-5 cm. diameter tuber under New Zealand Spring conditions. Color references are primarily to the RHS Colour Chart of The Royal Horticultural Society of London (RHS).

### THE PLANT

Classification:
   Botanical.—Zantedeschia spp.
   Common.—Calla Lily.
   Variety.—‘Goldilocks’.
Habit: Upright.
Plant:
   Height.—25-30 cm.
   Width.—25-41 cm.
   Number of shoots.—7-12.
   Number of shoots with flowers.—5-7.
Leaves:
   Length.—12-26 cm.
   Width.—5-8 cm.
   Shape of blade.—Lanceolate.
   Apex shape.—Acute, small angle.
   Base shape.—Obtuse.
   Spotting.—maculation of the leaf is present.
   Extent of maculation.—Average.
   Color of blade.—Medium green (RHS 137C).
Petiole (leaf stalk):
   Length.—10-15 cm.
   Color.—Base is light green (RHS 143B) with very weak purple (RHS N187A).

### THE INFLORESCENCE

Scape:
   Length.—21-29 cm.
   Thickness.—Medium.
   Color.—Light green (RHS 145B) with medium, purple mottling (RHS N187A) at base.
Spathe:
   Shape.—Cylindric, with medium overlapping.
   Length.—4-6 cm.
   Longest diameter.—(across top) 45-55 mm.
   Shortest diameter.—(across top) 30-45 mm.
   Primary color.—Yellow (RHS 5B).
   Texture.—Medium.
   Color.—Outside yellow (RHS 4C); innerside consistent yellow (RHS 5B).
Spadix:
   Height.—20-35 cm.
   Width.—3-5 cm.
   Color.—Yellow-orange (RHS 13A).
   Throat.—Purple (RHS N186B) blotch.
   Size of blotch in throat.—Medium.

1. A new and distinct cultivar of Zantedeschia plant as shown and described herein.
